(19) United States
(12) Patent Application Publication
(10) Pub. No.: US 2010/0291541 A1
(43) Pub. Date: Nov. 18, 2010

(54) BACTERIOPHAGE IMMOBILIZATION FOR BIOSENSORS

(76) Inventors: Stephane EVOY, Edmonton (CA); Amit SINGH, Edmonton (CA); Nicholas R. GLASS, Edmonton (CA)

Correspondence Address: Lambert Intellectual Property Law, Suite 200, 10328 - 81 Avenue, Edmonton, AB, T6E 1X2, CA

(21) Appl. No.: 12/466,089

(22) Filed: May 14, 2009

(51) Int. Cl. C12Q 1/70; C12N 7/00; C12M 1/34

(52) U.S. Cl. 435/5; 435/235.1; 435/287.1

(57) ABSTRACT

A method is disclosed for anchoring a bacteriophage on a substrate, the bacteriophage having a phage amine moiety, the method comprising: producing a free amine terminal moiety on the substrate by chemical modification of the substrate; activating the free amine terminal moiety with a cross-linking agent to produce an active functional group to couple to the phage amine moiety; and anchoring the bacteriophage to the substrate using the active functional group. A sensor is also disclosed comprising: a substrate; an anchor group attached by chemical modification to the substrate and having an active functional group produced by the activation of a free amine terminal moiety; and a bacteriophage having a phage amine moiety coupled to the active functional group to anchor the bacteriophage to the substrate.

### TECHNICAL FIELD

The work describes a chemical attachment method of bacteriophages on surfaces, and a sensor made by such methods. This method can be used in applications that would benefit from the efficient immobilization of phages.

### BACKGROUND

Detection of pathogenic bacteria has been an area of prime interest in the field of food and water safety, public health and anti-bioterrorism. Conventional microbiological techniques take several days in order to culture small loads of bacteria from a sample to a detectable number. In addition, identifying the specific signature of a bacterium requires further biochemical and serological tests, which are costly, time consuming, and labour intensive. Alternative methods like Polymerase chain reaction (PCR) and Enzyme-linked Immunosorbent Assay (ELISA) suffer from low sample volume and associated problems.

Biosensing platforms have received increased attention as alternative methods for bacterial detection. Such platforms usually consist of three components: a biological recognition mechanism, a physical transduction platform, and a system to read the transduced signal. The transduction phenomenon can be optical, magnetic, thermoelectric, piezoelectric, electrochemical or mechanical in nature. A wide range of techniques such as quartz crystal microbalance (QCM), surface plasmon resonance (SPR), flow cytometry, amperometry, and micromechanical resonators have been extensively researched.

Bacteriophages, or phages, are viruses that bind to specific receptors on the bacterial surface in order to inject their genetic material inside the bacteria. These entities are typically of 20 to 200 nm in size. The injection of the phage nucleic acid into the bacterial cells allows the phages to propagate inside the host using the host's own replication machinery. The, replicated virions are eventually released, killing the bacterium and allowing the infection of more host cells. Phages generally recognize bacterium receptors through tail spike proteins. This recognition is highly specific and has thus been employed for the phage typing of bacteria.

Conventional biosensors developed have mainly relied on physical adsorption for the attachment of phages on the sensor surface. Adsoption approaches result in poor phage surface coverage, severely inhibiting the sensitivity of the platform. Thus, there is a need for an attachment process that improves the performance of these sensors.

### SUMMARY

A method is disclosed for anchoring a bacteriophage on a substrate, the bacteriophage having a phage amine moiety, the method comprising: producing a free amine terminal moiety on the substrate by chemical modification of the substrate; activating the free amine terminal moiety with a cross-linking agent to produce an active functional group to couple to the phage amine moiety; and anchoring the bacteriophage to the substrate using the active functional group.

A sensor is also disclosed comprising: a substrate; an anchor group attached by chemical modification to the substrate and having an active functional group produced by the activation of a free amine terminal moiety; and a bacteriophage having a phage amine moiety coupled to the active functional group to anchor the bacteriophage to the substrate.

A method is also disclosed for the anchoring of bacteriophages onto surfaces that leverages the phage's basic amine groups as an anchor ligand. This anchoring is achieved by i) a chemical modification of the surface in order to produce a free amine terminal moiety on it, ii) activation of the amine terminal by a cross-linking agent in order to obtain an active functional group to couple to the phage amine group, and iii) attachment of the phages to the activated surface using the free amine groups present on their surface.

A method of chemical attachment of bacteriophages on different substrates has also been provided. The method may include attaching different types of phages on to different transducing platforms for developing biosensors for bacterial detection. The approach may use the phage's basic amine (NH₂) groups as opposed to their acidic carboxylic (COOH) group. This is accomplished by using cross-linking agents including, but not limited to, aldehydes, succinamides, sulfonates, or azides in order to latch onto the phage amine groups. This approach represents a distinct advantage given the preponderance of amines groups in most phages. As a result, it is equally applicable for the phages associated to a wide number of pathogens including, but not limited to, E. coli, Camphylobacter, Listeria, and Salmonella. Through proper selection of the terminal group (eg. thiol, silane, aldehyde, etc. . . . ), the immobilization can be performed a on a wide variety of materials such as gold, silver, copper, silicon nitride, silicon carbonitride, glass, and cellulose. Chemical attachment of phages onto a surface yields better coverage and improved performance of a sensor than adsorption based attachment methods.

These and other aspects of the device and method are set out in the claims, which are incorporated here by reference.

### BRIEF DESCRIPTION OF THE FIGURES

Embodiments will now be described with reference to the figures, in which like reference characters denote like elements, by way of example, and in which:

FIG. 1 illustrates functionalization steps involved in immobilization of bacteriophages on a gold substrate.

FIG. 1A illustrates a sensor made from the method of FIG. 1.

FIGS. 2A-B are scanning electron microscope images of T4 wild type phages attached to an a) cysteine modified gold surface, and b) cysteamine modified gold surface. The scales correspond to 2 μm.

FIGS. 3A-D are SEM micrographs of phages immobilized on cysteine modified gold substrates at 25, 40, 50 and 55° C., respectively. All scale bars correspond to 2 μm.

FIGS. 3E-H are fluorescent images of the corresponding bacterial capture on the surfaces illustrated in FIGS. 3A-D, respectively. The bacteria were stained with SYTO stain prior to surface capture.

FIGS. 4A-B are scanning electron microscope images of the T4 wild type phages on a gluteraldehyde activated cysteine modified gold surface (shown in FIG. 4A) and a gluteraldehyde activated cysteine modified gold surface (shown in FIG. 4B). The scale bar correspond to 2 μm.

FIGS. 4C-D are fluorescent microscopic images of SYTO stained bacteria bound to phages immobilized on cysteine modified gluteraldehyde activated gold surfaces at 40× (shown in FIG. 4C) and cysteamine modified gluteraldehyde activated gold surface 64× magnifications (shown in FIG. 4D). The scale bars correspond to 50 μm.

FIGS. 5A-B are SEM micrographs of purified phages immobilized on gluteraldehyde activated cysteamine modified gold substrates at room temperature. The scale bars correspond to 500 nm.

### DETAILED DESCRIPTION

Immaterial modifications may be made to the embodiments described here without departing from what is covered by the claims.

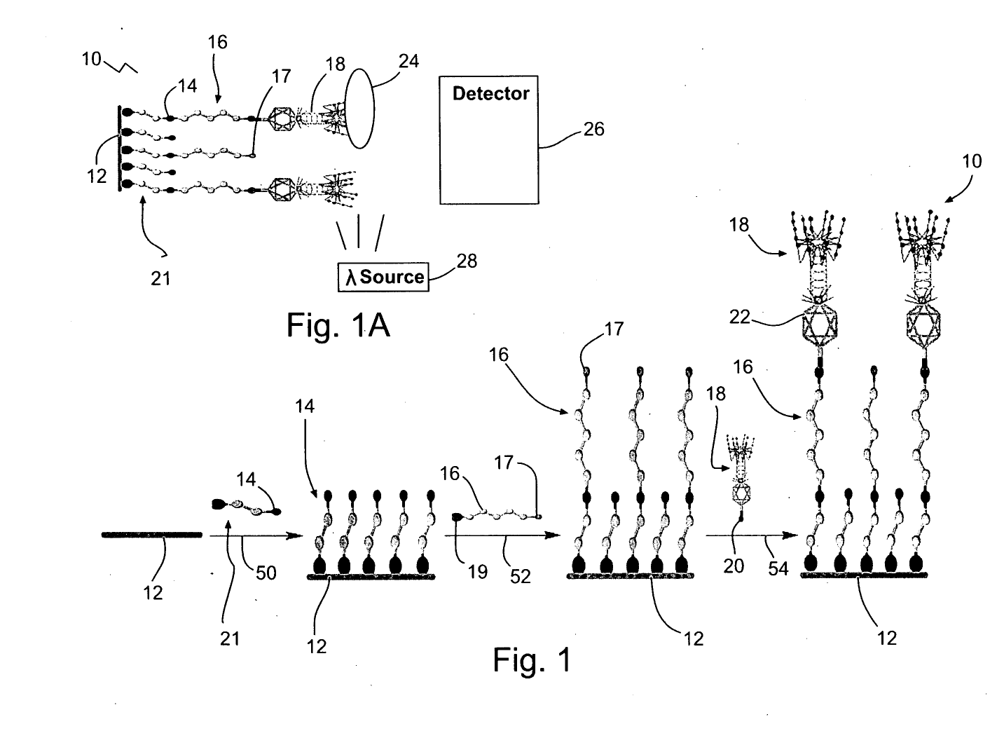

Referring to FIG. 1, a method for anchoring a bacteriophage 18 on a substrate 12 is illustrated. The bacteriophage 18 has a phage amine moiety 20, for example an amine containing functional group located on the head 22 of the phage 18. The substrate may be a suitable substrate for a biosensor, and may be curved, flat, or other suitable configurations.

Referring to FIG. 1, in stage 50, a free amine terminal moiety 14 is produced, for example deposited or chemically added, on the substrate 12 by chemical modification of the substrate 12. The substrate may be any suitable substrate and may for example comprise one or more of gold, silver, copper, nickel, cobalt, iron silicon, silicon nitride, silicon carbonitride, glass, polymer, ceramic, and cellulose. In the examples detailed herein, gold surface was taken as a model surface for immobilization. The substrates may be fabricated by known methods, for example fabricating the gold substrate using a piranha cleaned 3″ silicon (100) wafer by sputtering a 5 nm thick chrome adhesion layer followed by a 20 nm thick layer of gold. The gold coated wafers in the examples disclosed herein were diced into 5 mm×7 mm rectangular pieces using a diamond tip pen. The substrates were then sonicated for 5 min in acetone followed by cleaning in isopropanol and ethanol for 5 min each. They were finally rinsed in Milli Q water for 5 min prior to their surface functionalization.

The free amine terminal moiety 14 may be produced on the substrate 12 by chemical attachment of an anchor group 21 (shown in FIG. 1A), which may comprise any suitable anchor group for example one or more of a thiol, silane, carboxylic acid, and aldehyde. The anchor group may comprise an amino acid, for example cysteine, cysteamine, or histidine. Stage 50 may be carried out by exposing the washed substrates to a solution of cysteamine hydrochloride (cysteamine is shown as the anchor group in FIG. 1) for a suitable amount of time, for example 20 hours at room temperature. In the example disclosed, the solution of cysteamine hydrochloride is 50 mM, although other concentrations are possible depending on the specific implementation. A suitable temperature may be used, for example 40 and 60° C. The substrate may then be washed twice in deionized water and used. Stage 50 may be carried out by suitable methods, including for example one or more of chemical vapour deposition, surface functionalization, and chemical or electrochemical methods.

Referring to FIG. 1, in stage 52, the free amine terminal moiety 14 is activated with a cross-linking agent 16 to produce an active functional group 17 to couple to the phage amine moiety 20. The aim of the active functional group 17 is to capture free amine group on the phages for immobilization. Crosslinking agents 16 may have an amine-capturing active functional group and a substrate anchoring functional group 19 on another end for coupling to the free amine terminal moiety. The substrate anchoring functional group and the active functional group may be the same type of group. The active functional group 17 may be for example an active ester such as a succinimidyl ester (SE), a sulfosuccinimidyl ester (SSE), a tetrafluorophenyl ester (TFP), and a sulfodichlorophenol ester (SDP), an isothiocyanate (ITC), and a sulfonyl halide such as sulfonyl chloride (SC). Other groups may be used for the active funcional group, for example an ester, an aldehyde, a succinamide, a sulfonate, an ester, an isothiocyanate, a halide, an azide, a dichlorotriazine, an aryl halide, an aldehyde, an activated acyl derivative, an active cyclic acyl compound, an acyl sulphonamide, and an acyl azide to name a few. The cross-linking agent 16 may replace the free amine terminal group with the active functional group 17. The phage amine moiety, for example a free terminal amine moiety, is understood to be suitable and available for coupling with the active functional group 17.

Activation may be carried out by suitable methods, for example by incubation in solution of gluteraldehyde for a suitable amount of time at a suitable temperature. Examples of suitable respective concentrations, amounts of time, and temperatures in the preceding sentence include 2% solution (v/v), 1 hour, and room temperature. Afterwards, the substrate may be cleansed, for example by washing twice with deionized water for 5 min each. The modified substrates may then be used for immobilization of any bacteriophage, such as wild type T4 bacteriophages.

Referring to FIG. 1, in stage 54, the bacteriophage 18 is anchored to the substrate 12 using the active functional group 17. As shown, plural bacteriophages may be anchored to the substrate 12.

Bacteriophages may be obtained by any of various suitable conventional methods. For example, amplification of wild-type T4 phages may be achieved using the established protocol detailed below. 100 μl of 10² plaque forming units (pfu) of phage were incubated in 4 mL of fresh log-phase E. coli EC12 bacterial culture for 15 min at room temperature. The mixture was then added to 250 mL of LB media and was incubated at 37° C. in a shaking incubator for 6 h. The solution was then centrifuged at 4000 g for 10 min in order to pellet the bacteria. The supernatant was filtered through a 0.22 μm filter to remove any remaining bacteria in the solution. Ultra-centrifugation was performed at 55 000 rpm for 1 h to pellet the bacteriophages from the filtered supernatant. SM buffer (1.5 mL) was added to the phage pellet and the solution incubated overnight at 4° C. to allow the phages to resuspend. Bacterial enumeration was done by plate count method and was expressed in cfu/mL while the phage count was performed using the soft agar overlay technique and expressed in pfu/mL. Further, the phage solution was purified on a Sephacryl S-1000 SF (GE Life Sciences) to get rid of bacterial contaminant proteins. The purified phage solution was then checked for surface immobilization and the results were compared to that of unpurified phage solution.

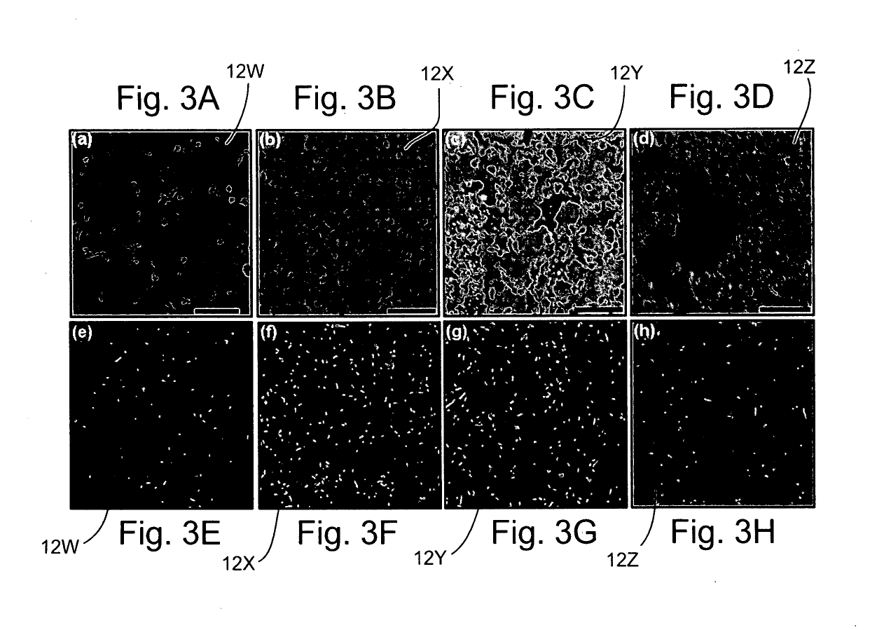

The bacteriophages 18 used in stage 54 may be purified or unpurified. An exemplary immobilization of unpurified phages is now described. An unpurified phage titre was adjusted to 10¹² pfu/mL by dilution with SM buffer and the same concentration was used for all the phage immobilization work. The surface modified gold substrates 12 may be immersed in the phage solution for a suitable time and a suitable temperature, for example 20 h and room temperature (25° C.), respectively. The effect of temperature on the surface density of phages on the substrate was also studied by immobilizing them at 40° C., 50° C., 55° C. and 60° C., see discussion below with regards to FIGS. 3A-H. Substrates 12 may be thoroughly washed in 0.05% (v/v) Tween-20 solution in SM buffer, washed twice in SM buffer and deionized water, and dried under dry nitrogen. All the washing steps may be performed at room temperature on an orbital shaker. The surfaces may be inspected using a Hitachi S-4800 (Tokyo, Japan) scanning electron microscope (SEM). The density of the phages on the substrates may be calculated based on the counts from these SEM images.

Exemplary immobilization of purified phages is now described. The purified phages may be immobilized on the activated surface. The surfaces may be washed, for example in acetone, isopropanol, ethanol and water for 5 min each prior to surface functionalization. The cleaned surfaces may be incubated in for example cysteamine at room temperature, 40 and 60° C. for 30 min followed by washing in water. The cysteamine self-assembled monolayer may be activated in for example 2% aqueous solution of gluteraldehyde for 30 min and was then washed in water for 5 min. The activated surfaces may then be incubated in purified phage solution for 30 min at room temperature, 40 and 60° C. The surfaces were finally washed in 0.05% (v/v) Tween-20 solution in SM buffer, washed twice in SM buffer and deionized water, and dried under dry nitrogen. All the washing steps were performed at room temperature on an orbital shaker. Methods with purified or unpurified phages may be carried out in a similar fashion.

The bacteriophage used may comprise a bacteriophage specific to a particular type of bacteria, for example one or more of E. coli, Salmonella typhimurium, Campylobacter jejuni, and Listeria sp. More than one type of bacteriophage may be used on a substrate, in order to detect for more than one type of bacteria. Any suitable phage may be used. Referring to FIG. 1A, the method may further comprise a stage where the presence of bacteria 24 coupled to the bacteriophage 18 anchored to the substrate 12 is detected, for example with a suitable detector 26. Detecting may further comprise exposing the bacteriophage 18 anchored to the substrate 12 to a sample comprising bacteria 24, so that any bacteria 24 specific to phages 18 may by coupled to the phages 18 and remain on the surface of the substrate 12. The presence of bacteria 24 connected to the bacteriophage 18 anchored to substrate 12 may then be detected, for example using fluorescence. Fluorescence spectroscopy may be carried out using a light source 28 positioned to irradiate substrate 12 with light of a particular wavelength known to induce fluorescence from bacteria 24. In these embodiments, detector 26 may be positioned to detect only fluoresced light released from bacteria 28. Detector 26 may support the substrate in detection position.

Referring to FIG. 1A, a sensor 10 is illustrated, comprising a substrate 12. An anchor group 21 is attached by chemical modification to the substrate 12 and has an active functional group 17 produced by the activation of a free amine terminal moiety 14. It should be understood that active functional group 17 may not be available or active for further reaction after coupling to the phage amine moiety 14. Sensor 10 further comprises a bacteriophage 18 having a phage amine moiety 20 (shown in FIG. 1) coupled to the active functional group 17 to anchor the bacteriophage 18 to the substrate 12. The active functional group 17 may be located on a cross-linking agent 16 coupled to the free amine terminal moiety 14. In this embodiment, bacteriophage 18 is anchored by connection to active functional group 17 of cross-linking agent 16, the cross-linking agent 16 being connected to free amine terminal moiety 14 of the substrate 12. This may be accomplished using suitable methods such as the ones disclosed herein. A suitable detector 26 for detecting the presence of bacteria 24 coupled to the bacteriophage 18 anchored to the substrate 12 may be provided. In other embodiments, the substrate 12 may be visually observed using suitable methods, such as observation under a fluorescence microscope. Sensor 10 may be used to detect the presence of a type of bacteria 24 specific to the anchored phages 18 used in an efficient manner.

The amplification of host and control bacteria used in the disclosed exemplary cases is now described. Fresh cultures of E. coli host (EC12) and controls (6M1N1, NP30 and NP10) were grown in LB medium to obtain a bacterial density of 10⁸ cfu/mL. The culture (1 mL) was then centrifuged and resuspended in 1 mL of 5% TSB (tryptic soy buffer) in 0.15 M NaCl solution. The bacteria were then stained with SYTO 13 for 15 min to be analyzed by fluorescence microscopy. The phage immobilized substrates 12 may be immersed in the bacterial solution for a suitable amount of time, for example 30 min. The substrates may then be washed, for example in TSB to remove excess stain. Further washing may be done, for example thorough washing in 0.05% tween-20 solution in TSB to remove loosely bound bacteria. Even further washing in TSB may be performed. The substrates 12 may then be observed under a fluorescence microscope. Samples may be washed under shaking condition at room temperature on an orbital shaker. An Olympus IX81 microscope (Tokyo, Japan) equipped with an FITC filter and a Roper Scientific Cool-Snaps HQ CCD camera (Duluth, Ga., USA) may be used to record the fluorescence images. Each fluorescent dot counted may be considered as a bacterium to establish the bacterial density on the surface. The captured bacteria may also be fixed in 2% aqueous solution of gluteraldehyde for 1 hr and imaged in SEM to calculate the surface density.

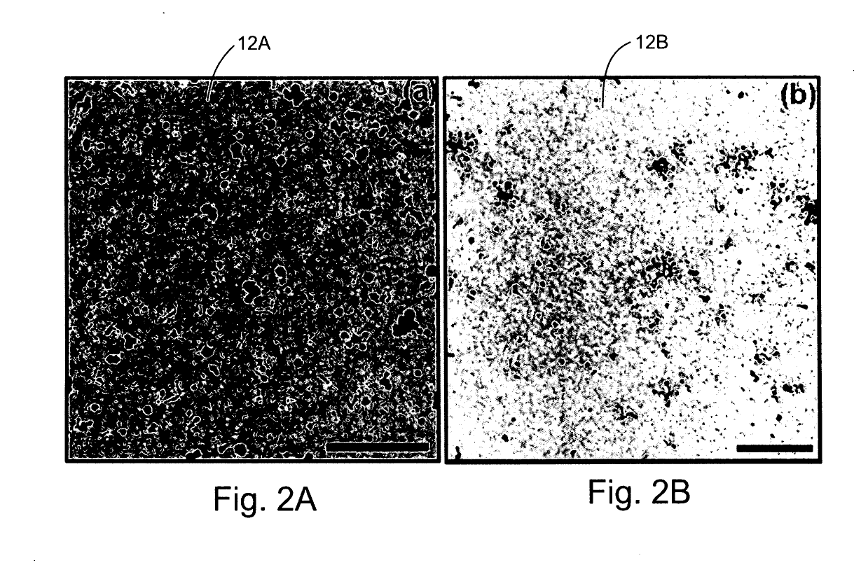

The results of exemplary cases carried out are now described. Referring to FIGS. 2A,B, surfaces modified with cysteine (FIG. 2A, substrate 12A) and cysteamine (FIG. 2B, substrate 12B) at room temperature yielded phage surface densities of 3.38±0.1 μm^{˜2} and 3±0.4 μm^{˜}2, respectively, a seven-fold increase over physical adsorption. Referring to FIG. 2A, the cysteine modified surface 12A yielded a bacterial density of 3.98±0.15 bacteria/100 μm².

Referring to FIG. 3A-D, the attachment of phages with cysteine was then performed at varying temperatures of 25° C., 40° C., 50° C. and 55° C. to further facilitate and optimize coverage. FIGS. 3A-D are electron micrographs of the resulting phage attachment on respective substrates 12W, 12X, 12Y, and 12Z, respectively. In turn, FIGS. 3E-H illustrate the respective fluorescence images from FIGS. 3A-D of the related bacterial capture. Referring to FIGS. 3A and 3E, immobilization performed at 25° C. yielded a phage density of 3.4±0.5 μm⁻² and a bacterial capture of 2.1±0.2 bacteria/100 μm². Referring to FIGS. 3B, F, these values increased to 7.2±0.7 μm⁻² and 4.8±0.7 bacteria/100 μm² when the phage immobilization was performed at 40° C. Referring to FIGS. 3C and 3D phages tended to cluster on the surface at immobilization temperatures of 50° C. and 55° C., complicating the evaluation of their density. This clustering also significantly reduced the bacterial capture, as evidenced by respective FIGS. 3G and 3H. Phages immobilized at temperatures of 60° C. and higher in the study done lost their capturing abilities altogether. Bacterial capture was therefore optimal in this example when the phage attachment was performed at 40° C. Negative control non-host strains showed no significant capture once again. All subsequent experiments were therefore performed under those conditions.

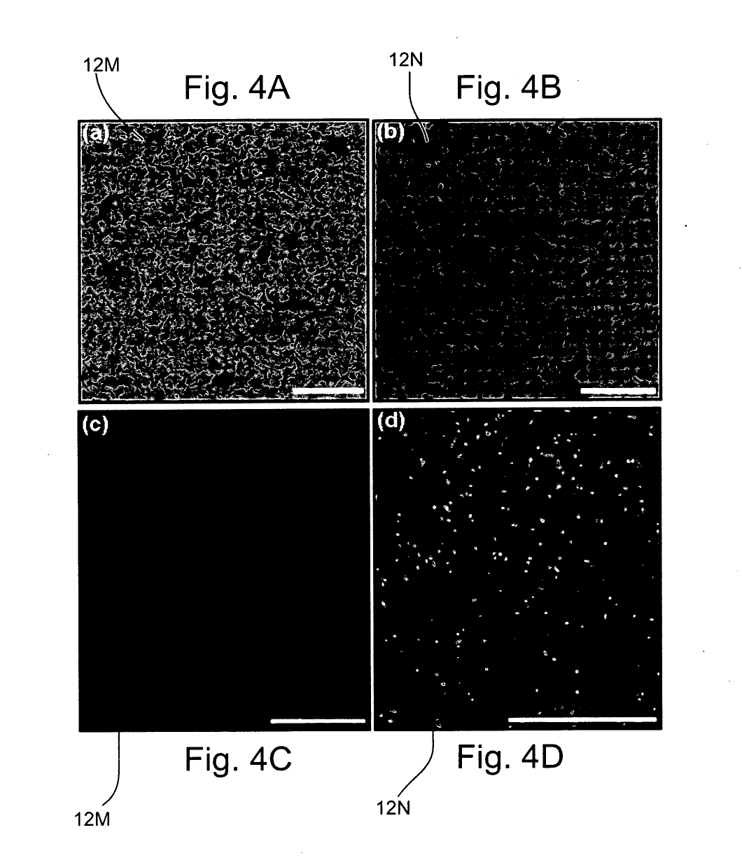

The effects of methods incorporating stage 52 were then tested. Referring to FIGS. 4A-B, Cysteine and cysteamine modified surfaces 12M and 12N, respectively, were activated with 2% aqueous solution of gluteraldehyde before anchoring in order to check for improvement in attachment efficiencies of phages. FIGS. 4A-B show micrographs of the resulting phage attachments. As a control, phages were physisorbed at 40° C. onto a bare gold surface and showed a density of 0.85±0.08 μm² (not shown). Referring to FIG. 4A, in contrast the density of the phages on a cysteine modified gluteraldehyde activated gold surface 12N was 17.1±0.9 phages/μm², a further five-fold improvement over non-activated cysteine (shown in FIG. 2a), and an overall 35-fold improvement over simple physisorption at room temperature. Referring to FIG. 4B, similarly, cysteamine modified gluteraldehyde activated surface 12N showed a density of 18±0.15 phages/μm², a 37-fold improvement over simple physisorption at room temperature. Referring to FIGS. 4C-D, respectively, these improved surfaces were subsequently checked for their ability to capture the host bacterium. Referring to FIG. 4C, the surfaces with phages attached with activated cysteine resulted in a capture density of 5.07±0.2 bacteria/100 μm², a 4-fold improvement over phages physisorbed at room temperature. Referring to FIG. 4D, the surfaces with phages attached with activated cysteamine resulted in a capture density of 11.9±0.2 bacteria/100 μm², a 9-fold improvement over phages physisorbed at room temperature. Once again, non-host strains did not show any significant binding on to the surface.

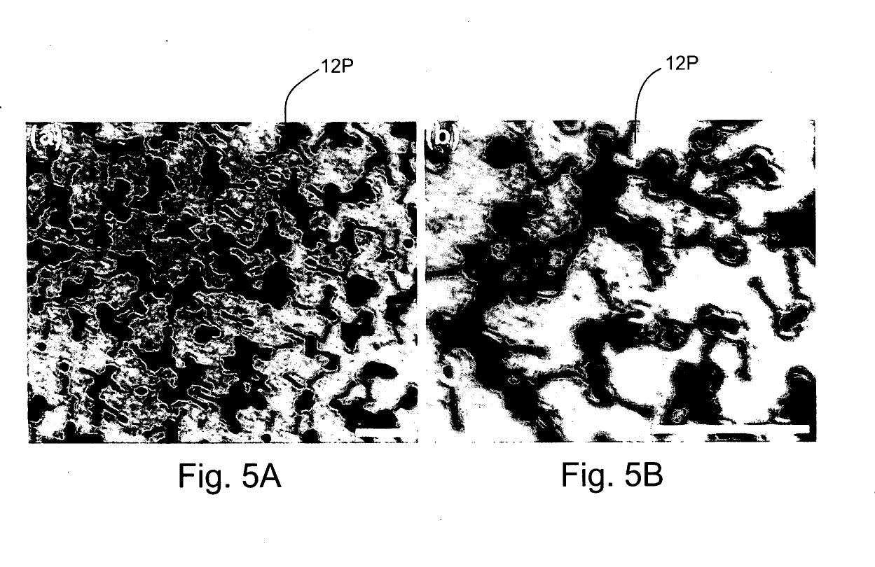

As mentioned, purified phages may be used in the anchoring stage 54. It was realized that a lot of unwanted proteins from the host bacterial culture (used for phage amplification) may contaminate the final phage solution. In order to further improve the density of phage immobilized on the activated surface, it may be necessary to remove the contaminant proteins from phage solution. A Sephacryl S-1000 SF (GE Life Sciences) column was used to remove these proteins from the phage solution. Referring to FIGS. 5A-B, the purified phage solution was used for immobilization on a gluteraldehyde activated cysteamine modified gold surface 12P. Referring to FIGS. 5A-B, a capture density of 46.83±1.6 phages/μm² was obtained, a 95-fold improvement over immobilization by simple physisorption of phages at room temperature. Further, the immobilization was effected within 30 min of incubation of the activated surfaces in the purified phage solution as opposed to an overnight incubation of 20 h in unactivated experiments. Thus, the purification of the phages results in a rapid immobilization with improved density on the surface.

The approach disclosed herein can be employed in numerous other suitable biosensing platforms such as for example microresonators, surface plasmon resonance, amperometric sensors, microcantilevers, and quartz crystal microbalance for example. In some embodiments, the phages used may be modified at least one of before and after the phage is anchored. The methods and apparatuses disclosed herein may be used for the biosensing of host bacteria. Anchoring refers to chemical attachment.

In the claims, the word “comprising” is used in its inclusive sense and does not exclude other elements being present. The indefinite article “a” before a claim feature does not exclude more than one of the feature being present. Each one of the individual features described here may be used in oe ene or more embodiments and is not, by virtue only of being described here, to be construed as essential to all embodiments as defined by the claims.

1. A method for anchoring a bacteriophage on a substrate, the bacteriophage having a phage amine moiety, the method comprising:
producing a free amine terminal moiety on the substrate by chemical modification of the substrate;
activating the free amine terminal moiety with a cross-linking agent to produce an active functional group to couple to the phage amine moiety; and
anchoring the bacteriophage to the substrate using the active functional group.

2. The method of claim 1 in which the substrate comprises one of gold, silver, copper, nickel, cobalt, iron silicon, silicon nitride, silicon carbonitride, glass, polymer, ceramic, and cellulose.

3. The method of claim 2 in which the substrate comprises gold.

4. The method of claim 1 in which the free amine terminal moiety is produced by chemical attachment of an anchor group.

5. The method of claim 4 in which the anchor group comprises one of a thiol, silane, carboxylic acid, and aldehyde.

6. The method of claim 4 in which the anchor group comprises one of cysteine and cysteamine.

7. The method of claim 1 in which the free amine terminal moiety is produced on the substrate by one or more of chemical vapour deposition, surface functionalization, and chemical or electrochemical methods.

8. The method of claim 1 in which the bacteriophage comprises a bacteriophage specific to one of E. coli, Salmonella typhimurium, Campylobacter jejuni, and Listeria sp.

9. The method of claim 1 in which the cross-linking agent comprises one or more of an aldehyde, a succinamide, a sulfonate, an ester, an isothiocyanate, a halide, and an azide.

10. The method of claim 9 in which the cross-linking agent comprises gluteraldehyde.

11. The method of claim 1 further comprising detecting the presence of bacteria coupled to the bacteriophage anchored to the substrate.

12. The method of claim 11 in which detecting further comprises:
exposing the bacteriophage anchored to the substrate to a sample comprising bacteria; and
detecting for the presence of bacteria coupled to the bacteriophage using fluorescence.

13. The method of claim 1 used to anchor plural bacteriophages to a substrate.

14. The method of claim 1 in which the bacteriophage used in the anchoring stage is purified.

15. A sensor comprising:
a substrate;
an anchor group attached by chemical modification to the substrate and having an active functional group produced by the activation of a free amine terminal moiety; and
a bacteriophage having a phage amine moiety coupled to the active functional group to anchor the bacteriophage to the substrate.

16. The sensor of claim 15 in which the active functional group is located on a cross-linking agent coupled to the free amine terminal moiety.

17. The sensor of claim 15 further comprising a detector for detecting the presence of bacteria coupled to the bacteriophage anchored to the substrate.

18. The sensor of claim 15 in which plural bacteriophage are anchored to the substrate.
